(19) United States
(12) Patent Application Publication
(10) Pub. No.: US 2011/0097817 A1
(43) Pub. Date: Apr. 28, 2011

(54) REACTION DEVICE FOR ANALYZING BIOLOGICAL SAMPLES AND RELATIVE METHOD

(75) Inventors: Paolo Galiano, Padova (IT); Francesco Frappa, Camino Al Tagliamento (IT)

(73) Assignee: ALIFAX SPA CON SOCIO UNICO, Polverara (IT)

(21) Appl. No.: 12/994,591

(22) PCT Filed: May 22, 2009
(86) PCT No.: PCT/EP2009/056249

§ 371 Date: Nov. 24, 2010

(30) Foreign Application Priority Data
May 27, 2008 (IT) UD2008A000127

(51) Int. Cl. G01N 33/53; G01N 35/00

(52) U.S. Cl. 436/501; 422/63; 436/43

(57) ABSTRACT

A reaction device and method provided with a reaction cell in which, in sequence, a plurality of biological samples, liquid, semi-liquid or mixed, of analogous or different type, are subjected to a reaction for a determinate analysis. The device comprises a first dispenser element which introduces a predetermined quantity of the biological sample inside the cell; a plurality of microvalve dispensers, each of which nebulizes a determinate microvolume of a desired reagent inside the cell, wherein the type of reagent and the amount of the microvolume of reagent are chosen as a function of the specific reaction to be carried out, selected from a group comprising reactions for analyses of a chemical-biological type, such as immunological or coagulative reactions, or of a chemical-physical type; a mixer element which mixes the biological sample with the selected reagent; a second dispenser element which sends to analytical measurement a determinate quantity of the biological sample after reaction with the selected reagent; washing members and discharge members, able respectively to wash the inside of the cell and to discharge the content of the cell, so as to prepare the cell for a subsequent reaction; an electronic processor which commands and controls, in the desired times and modes, the functioning of both the first dispenser element which dispenses the sample, of the microvalve dispensers which dispense the reagents, and of the mixer element, and also of the second dispenser element which sends the reacted sample for measurement, and of the washing members and discharge members, as a function of the type of reaction to be performed and the type of biological sample to be analyzed, so that their functioning is synchronized and automated according to pre-determined memorized reaction programs, selectable by an operator.

### FIELD OF THE INVENTION

The present invention concerns a reaction device and the relative method, used in an analysis apparatus for biological samples, such as blood, liquids of the blood (plasma, serum), cerebrospinal fluid, pleural fluid, peritoneal fluid, pericardial fluid, amniotic fluid, seminal fluid, biological secretions of the physiological channels (vaginal, ocular, . . . ), urine, feces and other. The reaction device according to the present invention allows to carry out one or more sequential reactions of various types, for example chemical-biological (including coagulation), chemical-physical, immunological, agglutination/aggregation or chemical-physical reactions/transformations on a sample, suitably dispensed by a dispenser device, for the subsequent measurement of the reacted sample for the desired or required analysis, providing the result of the analysis.

### BACKGROUND OF THE INVENTION

Various types of instruments are known for different types of biological samples and for different analytes.

The flow of work in a modern laboratory uses automatic instruments (cell-sorters) which prepare specific containers, according to the type of sample or anticoagulant contained in the test tubes, to be sent to the specific dedicated instrument or dedicated reaction unit, which does the analysis.

Therefore, there are dedicated instruments downstream of said cell-sorter which receive samples of whole blood or, after centrifugation, samples of plasma or serum or urine etc., and subject them to the desired reactions, propedeutic to the determinate analysis.

Laboratories that are not supplied with said cell-sorters separate and sort the test tubes manually, and load them manually into the instruments dedicated to this type of sample.

This has the disadvantage that, in a laboratory that analyzes different types of biological sample in order to effect different types of reaction for various measurements of a chemical-biological and chemical-physical type, it is necessary to use several reaction units, each one associated with a determinate analytical machine.

Each known analytical machine in turn occupies a pre-determined physical space, has a determinate procedure for use, requires determinate and specific reagents, and occupies a determinate number of operators.

As is obvious, this leads to a high waste of resources in terms of space, time, resources, raw materials and economic cost.

Purpose of the present invention is to achieve a reaction device and perfect a relative method, that allows to effect a plurality of different reactions, both of the chemical-biological type and of the chemical-physical type, on a plurality of different types of biological sample, quickly, automatically, efficiently, safely and economically.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purpose, a reaction device for analyzing a biological sample is used for biological samples, liquid, semi-liquid or mixed, such as: blood, liquids of the blood (plasma, serum), cerebrospinal fluid, pleural fluid, peritoneal fluid, pericardial fluid, amniotic fluid, seminal fluid, biological secretions of the physiological channels (vaginal, ocular, . . . ), urine, feces dissolved in liquid and other.

The device according to the present invention is provided with a reaction cell in which a plurality of liquid, semi-liquid or mixed biological samples, of analogous or different type, are subjected in sequence to a reaction for a determinate analysis.

According to a characteristic feature of the present invention, the reaction device comprises a first dispenser element able to introduce a predetermined quantity of the biological sample inside the cell and a plurality of microvalve dispensers, each of which is able to nebulize a determinate microvolume of a desired reagent inside the cell. The type of reagent and the amount of the microvolume of the reagent are chosen as a function of the specific reaction to be made, or of the type of analysis, selected from a group comprising: reactions for analyses of a chemical-biological type, such as immunological or coagulative reactions, or of a chemical-physical type.

The reaction device also comprises a mixer element, which is able to mix the biological sample with the reagent selected, a second dispenser element able to send for analytical measurement a determinate quantity of the biological sample after the reaction with the selected reagent, and washing and discharge means, able respectively to wash the inside of the cell and to discharge the content of the cell in order to prepare the cell for the subsequent reaction.

The reaction device according to the present invention also comprises electronic processing means able to command and control, in the times and modes desired, at least the functioning of the first dispenser element which dispenses the sample, the microvalve dispensers which dispense the reagents and the mixer element, and also the second dispenser element that sends the reacted sample for measurement, and also the washing and discharge means, according to the type of reaction to be made and the type of biological sample to be analyzed, so that their functioning is synchronized and automated according to predetermined and memorized reaction programs, selectable by an operator.

According to a variant, the invention comprises heating means able to heat and thermostat, for example for the purpose of incubation, the inside of the cell to a desired temperature value, correlated to the type of reaction to be made and according to a control signal received from the electronic processing means.

Advantageously, moreover, a blowing means is provided, able to blow in a selected fluid, for example air or oxygen, or an inert gas such as nitrogen or carbon dioxide, inside the cell, according to the type of reaction to be made and according to a control signal received from the electronic processing means.

According to an advantageous form of embodiment, a plurality of tanks is provided to contain a determinate reagent, each of which is connected to a corresponding microvalve dispenser.

Advantageously, a means to control the pressure is provided, to control and selectively vary the pressure inside the cell, according to the type of reaction to be made and according to a control signal received from the electronic processing means.

According to one form of embodiment of the present invention, the electronic processing means comprises an electronic memory in which the desired reaction programs have been memorized and a microprocessor able to execute said programs.

Advantageously, according to a selected work program, the microprocessor is able to transmit command signals at least to activate/de-activate the functioning and/or to regulate the quantities dispensed by the first and second dispenser element and by the microvalve dispensers.

Furthermore, according to a selected work program, the microprocessor can transmit command signals to activate/de-activate the functioning and/or to regulate the mixing power of the mixer element, according to the reaction and the biological sample.

According to a variant, the electronic processing means also comprises a timer, integrated with or separate from the microprocessor, by means of which the microprocessor is able at least to control and synchronize the functioning times of the first dispenser element that dispenses the sample, the microvalve dispensers that dispense the reagents and the mixer element, and also of the second dispenser element that sends the reacted sample for measurement, and also of the washing and discharge means.

Advantageously, the reaction device according to the present invention can be programmed for different mixing times and modes (slow, vortical, with air blown in by means of said blower means) and thus obtain mixing times and modes considered suitable for the type of sample and selectable for each type of analysis or test.

For example, this is advantageous for measuring erythrocyte sedimentation rate (ESR), which provides suitable result values with oxygenated blood with respect to the untreated blood sample. The present invention allows a better demonstration of the state of aggregation of the red corpuscles compared to samples made to sediment in conditions where there is little pre-analytical mixing or little oxygenation of the sample. Given the characteristic of red corpuscles to bind oxygen through the known mechanism of heme capture, their state of evident sedimentation determined by states of infection in which the known sedimentation of red corpuscles is shown, is expressed optimally when the blood sample is mixed and oxygenated.

Analogous and specific mixing times actuated through the programming of mixing times of the reaction device allow to distribute the reagents through a nebulizing process by means of the microvalve dispensers, which improves the mixing performance at the moment of dispensing.

This use of dispensing microvalves allows an exact and precise quantification of the dispensing and the dosing of the exit force of the reagent through the dosing nozzle, which provides, in the dispensing process, suitable types of mixing selectable according to quantity and exit times.

The microvalves thus allow a considerable saving in reagent, the use of which is optimized according to need, and also a high level of safety in the dispensing of the reagent, avoiding errors of imprecision.

The present invention therefore allows to selectively set a reaction with a single or multiple reagent according to the known types of reaction, instantaneous or sequential.

The present invention also allows to dispense the sample and distribute the phases of the reagents in sequential times or programmable according to the reactions in progress.

The present invention allows to effect a test or analysis allowing a differentiated mixing of sample and reagent, primary or extracting, followed by the exit of the second reagent at the desired times, allowing the pre-incubation steps to carry out the required reaction times.

The present invention allows to program the distribution of the reagents required according to volumes and times that can be programmed in the dispensing and differential incubation steps.

The present invention therefore allows a specific sequence of distributing the reagents and differentiated incubations required by the individual reagent introduced into the reaction cell.

The present invention is independent for every reagent distribution and sequential incubation because it is programmable in the mixing power and in the incubation time.

The present invention allows every individual reaction to be carried out in the differential or homogeneous times of the individual reagents required for a chemical immunological reaction detected with any type of method in existence, such as photometric, fluorometric and suchlike.

### BRIEF DESCRIPTION OF THE DRAWINGS

These and other characteristics of the present invention will become apparent from the following description of a preferential form of embodiment, given as a non-restrictive example with reference to the attached drawings wherein:

FIG. 1 is a schematic representation of a reaction device according to the present invention.

### DETAILED DESCRIPTION OF A PREFERENTIAL FORM OF EMBODIMENT

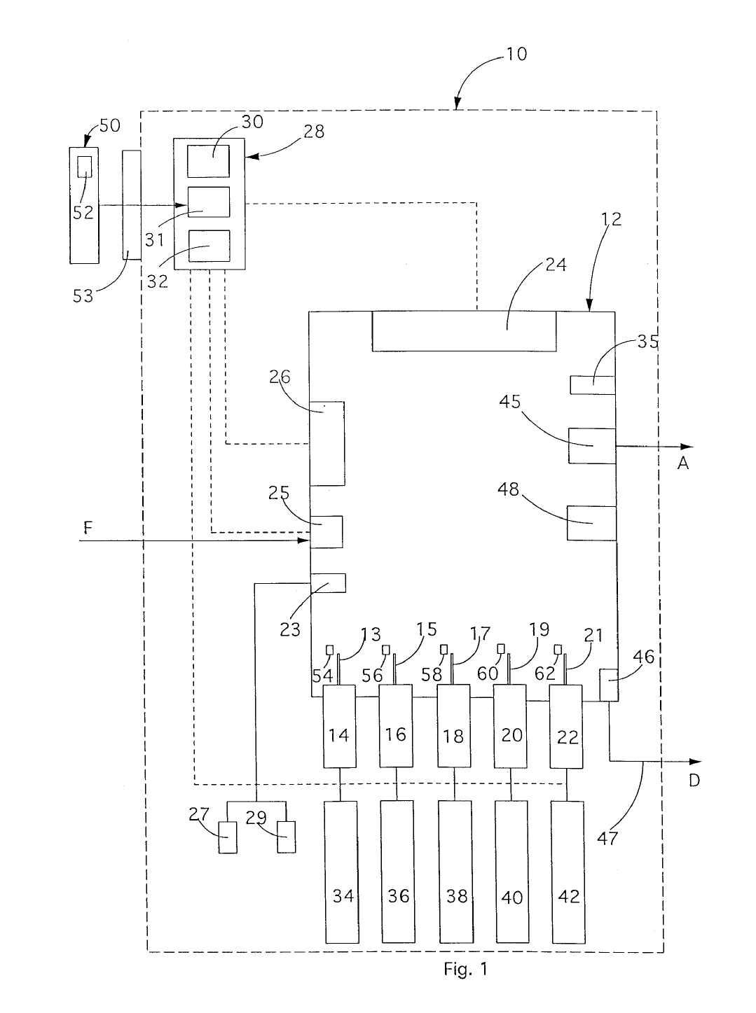

With reference to FIG. 1, a reaction device 10 according to the present invention to carry out one or more analytical reactions of a chemical-biological type, such as immunological, coagulative or chemical-physical type, such as lysis reactions, on a biological sample, of a liquid, semi-liquid or mixed type, comprises a reaction cell 12 provided with a dispenser element 25 to introduce, as indicated by the arrow F, the sample inside the cell 12, and five microvalves 14, 16, 18, 20 and 22 each able to dispense, inside the cell 12, a determinate reagent, according to the type of biological sample to be reacted, taken from a respective tank 34, 36, 38, 40 and 42.

The tanks can be thermostated, for example with Peltier cells, at about 4-8° C., so as not to compromise the efficiency thereof.

The dispenser element 25 is selected on each occasion according to the nature of the sample. For example a syringe can be used, a microvalve, a test tube, a spatula or other.

The reagents used can be, for example, latexes sensitized for a determinate antigen-antibody reaction of an immunological type, for example for C-reactive protein, rheumatoid factor RF, to assess fibrinogen, for streptolysin “O” (ASO test), or coagulative reagent, a lysis reagent, or chemical lysant, of whole blood, possibly to measure the erythrocyte sedimentation rate (ESR), anemia factor, viscosity of blood, corpuscle counters, reaction diluents (buffers), or other reagents for analyses on specific microorganisms, presence of hidden blood or other.

The device 10 can be of the independent type, or integrated into an analytical machine, not shown in the drawings, which also provides the pre-analytical preparation step, and the measurement of the reaction.

In particular, the microvalves 14, 16, 18, 20 and 22 are provided with a relative nebulizer nozzle 13, 15, 17, 19, 21 by means of which they are able to nebulize the desired reagent inside the cell 12, in order to increase the effectiveness of the subsequent mixing with the sample. Furthermore, the microvalves 14, 16, 18, 20 and 22 are also able to determine and dispense a desired and determinable microvolume of reagent (precise quantification of the reagent) and to dose the exit force of the reagent through the relative nozzle 13, 15, 17, 19, 21.

For example, each microvalve allows to dose volumes of about 10 nanoliters per impulse, delivering about 1000 drops per second.

By varying the number of total impulses the total volume delivered is easily varied and controlled.

When the concentration of the sample analyzed exceeds a determinate concentration threshold, the various microvalves 14, 16, 18, 20 and 22 allow to perform a second reaction with a greater dilution of the sample, delivering a determinate quantity of diluent (buffer), to prevent the “hook” effect, which is typical in this type of reaction, especially in serology.

If the dispenser 25 is a microvalve, it works in a coordinated manner with the microvalves 14, 16, 18, 20 and 22 so as to promote the mixing of the two liquids, with advantageous mixing results when the total delivery time coincides, that is, having established the volume which the two microvalves have to deliver, which can even be very different between the two, the delivery time will be the same for each microvalve and therefore their flow rate will vary.

Advantageously, the microvalves are oriented so as to deliver at a single common point, called the delivery focus.

The microvalves 14, 16, 18, 20 and 22 can be thermostated so as to deliver the volume of reagent necessary for the reaction at a standardized temperature of 37° C.±1.

Moreover, the dispensing of the reagents can be controlled optically by photometers 54, 56, 58, 60, 62, associated with the microvalves 14, 16, 18, 20 and 22, equipped with an emitter and a receiver 55, which substantially counts the drops actually delivered.

The reacted sample, that is, mixed with the reagent, or a part thereof, is sent by means of a dispenser 45 for analytical measuring, as indicated by arrow A.

In order to mix the selected reagent with the biological sample, a mixer element or agitator 24 is installed inside the cell 12, such as a vortex type system which is activated before the analytical cycle is started, so as to guarantee homogeneity of the reagent.

Furthermore, a heating element or thermostat 26, for example of the clip type, is provided for thermostating the inside of the cell 12, so as to determine the desired temperature conditions for the possible incubation of the sample-reagent mixture.

The reaction device 10 also comprises an electronic processor 28, provided at least with a memory 30, a microprocessor 31 and a timer 32.

The processor 28 is able to command and control the functioning of the microvalves 14, 16, 18, 20 and 22, the agitator 24 and also the thermostat 26.

In fact, the volumes and volume ratios that the microvalves and dispensers can deliver can be varied according to a determinate algorithm present in the microprocessor 31.

In particular, the microprocessor 31 is able to execute determinate reaction programs pre-loaded in the memory 30, which programs can be selected by the operator by means of a user interface such as a keyboard and display, according to the sample to be reacted and the type of reaction, and therefore of analysis, to which the sample is to be subjected.

The choice of the correct reaction program can also be made automatically, by providing that the device 10 or the analytical machine in which it is integrated is able to recognize autonomously, that is, without the intervention of the operator, the type of sample to be reacted and the desired reaction to be carried out in the specific case. Recognition may be made by means of a bar code or RFID tag, identifying the sample and/or rack that identify the type of sample (blood, plasma, serum, urine, feces) and relative test tubes contained therein.

These programs, divided therefore by type of reaction and of sample, consequently activate one or more of the microvalves 14, 16, 18, 20, 22, in order to introduce a determinate microvolume of reagent, they command the start of mixing by means of the agitator 24, for the desired mixing time, and possibly incubation, activating the thermostat 26 in this case depending on the necessary incubation period.

The reaction programs are also able to manage several reagents, by selectively activating the microvalves 14, 16, 18, 20, 22, in the case of reactions which provide sequential or simultaneous dispensing.

In particular, with the aid of the timer 32, the processor 28 is able to determine the desired times, in a sequential or programmable manner, of the mixing of the sample and the reagent, and also of the incubation of the mixture, according to the specific reaction to be carried out. The possibility of specific programming of the mixing times allows to distribute the nebulized reagents, which improves the mixing performance.

The thermostat 26 can also be used to determine a desired evaporation of the reagent from the cell 12, according to needs.

The introduction of the sample by means of the dispenser element 25 can also be commanded and controlled, possibly, by the processor 28, or can be determined manually.

According to a variant embodiment, each batch of reagents is associated with a card 50 with a magnetic band, or a microchip 52, or other analogous memorization device. In the microchip 52 of the card 50 a univocal identification is memorized, which indicates the specific batch of reagent. The univocal identification is used to constrain the use of the device 10 for the insertion and recognition of the card 50 in a suitable reader 53. The reader 53 reads the data of the card 50 and transmits them to the processor 28, which compares them with data already memorized inside it. Once it has recognized that the batch of reagents is precisely the one with which the device 10 can function, the processor gives consent and authorization for use of the device 10 and unblocks all the components. This allows to have a high level of safety and univocal use, because only the batches of reagents which comply with predefined operating standards of the device 10 and provided with a card 50 which identifies and attests this compliance can be used with success. The use of the device 10 is thus limited or constrained to the batches of reagent which include said recognition card 50.

It is advantageous to insert further data and information in the microchip 52, particularly data relating to the necessary volumes to be dispensed, the dilution/concentration of the reagent of the specific batch, or in any case the production specifications of the batch of reagents, so that it is not necessary to indicate or modify manually on each occasion the new volumes to the instrument (valves) for good functioning. It will be the card 50, suitably instructed, which will command the volumes chosen of the specific production batch.

In fact it is known that, particularly in immunological reactions, the exact dosage of the reagents is crucially important in order to obtain calibration curves in line with the normal-pathological reference values so as to obtain results that vary as little as possible with respect to the control standards available on the market, so as to prevent unreliable measurements. In particular, it is known that in many immunological reactions the volumetric quantity of the reagents referable to antigens and antibodies in solution or to coated antigens and antibodies with particles of various type, such as latexes or colloidal gold, can vary in their quantity defined by the individual production batch in order to maintain response standards with reference to the calibration curve inasmuch as, either due to a change in avidity of the antibody, or to purification variables of the antigens, these quantities can or must be changed for each production batch.

Therefore, the card 50, having already memorized the optimized volumes for the specific production batch, allows to distribute the calibrated volume with respect to the control standards and the calibration curve in order to obtain reliable results.

The card 50 can instruct the processor 28 precisely on the modes of microdosing the various reagents (in particular the correct volumes). The processor 28, according to the data read by the card 50, can correctly command automatically the opening and closing of the microvalves 54, 56, 58, 60 and 62. In this way we obtain a balanced microdispensing in the volumes to be used, optimized for the specific volumes of the individual production batches. This allows a highly repeatable and reliable response standard and avoids the need to make the operator/technician responsible for the analysis work manually, that is, without having to modify the dosage of the microvalve manually, reducing risks of errors and making the analysis procedure quicker.

Furthermore, the microchip 52 may also have memorized in it data relating to the correct and necessary reaction parameters. In particular, apart from the times and temperatures of the reactions, the specific calibration curve may be memorized for each production batch for the execution of each individual reaction and analysis, without further waste of reagent for the execution of said analyses, or using a single point of the curve as a reference of good reliability of the production batch of reagents.

The microchip 52 functions only for each production batch of reagents with which an identification code is associated for the correct execution of the test, and is not re-usable.

A discharge pipe 46, selectively openable/closable upon command from the processor 28 and which can be connected with an intake line 47 to facilitate expulsion, allows to discharge a determinate reagent, as indicated by the arrow D, for example in the case of reactions that require a primary reagent and a secondary or extracting reagent, with discharge of the second reagent at the desired times.

The discharge pipe 46 can also be used at the end of the reaction to discharge all the content of the cell 12, when it is necessary to clean the cell 12, in order to proceed with another reaction of the same type, or a different type, with the same type of sample, or a different sample. In this case, a dispenser device 48 for a washing liquid or fluid provides to introduce the liquid or fluid, once it has received a signal to do so from the processor 28, which can be agitated to obtain an effective washing of the whole, and is finally discharged.

In addition, the cell 12 can be provided with a pressure control device 23, which can be connected, selectively and according to a relative signal transmitted by the processor 28, to a pump 27 or an aspirator 29, in order to selectively put the inside of the cell 12 under pressure or vacuum, according to the reaction needs.

Furthermore, the cell 12 can be provided with a blower device 35 able to blow a fluid, gas or vapor inside the cell 12, such as for example air, oxygen, inert gases such as carbon dioxide, nitrogen, to determine a desired composition of the atmosphere inside the cell 12 (protected and/or modified atmosphere, rich in air or oxygen or other). The blower device 35 can advantageously operate in synchrony with the agitator 24. For example, this is advantageous because determinate reactions propedeutic to the measurement of the erythrocyte sedimentation rate (ESR) provide correct results preferably with oxygenated and/or effectively mixed blood.

The device 23 is also advantageously commanded and controlled by means of the processor 28, to determine the desired or required pressure value in the cell 12.

It is clear that the functions of the blower device 23 and the dispenser device 48 of the washing liquid can be integrated in a single device that selectively introduces the desired fluid, gas, vapor or liquid according to the various steps of the reaction, all this being commanded by the processor 28.

The device 10 functions as follows.

There is a first step in which a predetermined quantity of the biological sample is introduced inside the cell 12, by means of the dispenser 25, and a second step in which a determinate microvolume of a desired reagent is nebulized inside the cell, by a selected one of the microvalves 14, 16, 18, 20, 22. The type and amount of the reagent are chosen according to the specific reaction to be carried out.

There is then a third step in which the biological sample is agitated and homogenized with the selected reagent, by activating the agitator 24.

Once the biological sample has mixed and reacted with the selected reagent, there is a fourth step in which it is sent for analytical measurement by means of the dispenser.

Finally, to prepare the cell 12 for a subsequent reaction, possibly with a different sample and/or reagent, there is a fifth step in which the cell 12 is washed and the content of the cell 12 is discharged, respectively by means of the dispenser 48 of washing liquid and the discharge pipe 46.

The whole is commanded and controlled, in the desired times and modes, by means of the processor 28, according to the type of reaction to be carried out and the type of biological sample to be analyzed, so that the functioning is synchronized and automated on the basis of the predetermined memorized reaction programs, selectable by the operator.

It is clear that modifications and/or additions of parts and/or steps may be made to the reaction device 10 and the relative method as described heretofore, without departing from the field and scope of the present invention. It is also clear that, although the present invention has been described with reference to specific examples, a person of skill in the art shall certainly be able to achieve many other equivalent forms of reaction device for analyzing biological samples and relative method, having the characteristics as set forth in the claims and hence all coming within the field of protection defined thereby.

1. A reaction device provided with a reaction cell in which, in sequence, a plurality of biological samples, liquid, semi-liquid or mixed, of analogous or different type, are subjected to a reaction for a determinate analysis, the reaction device comprising:
a first dispenser element able to introduce a predetermined quantity of the biological sample inside the cell;
a plurality of microvalve dispensers, each of which is able to nebulize a determinate microvolume of a desired reagent inside the cell, wherein the type of reagent and the amount of the microvolume of reagent are chosen as a function of the specific reaction to be carried out, selected from a group comprising: reactions for analyses of a chemical-biological type, such as immunological or coagulative reactions, or of a chemical-physical type;
a mixer element which is able to mix the biological sample with the selected reagent;
a second dispenser element able to send to analytical measurement a determinate quantity of the biological sample after reaction with the selected reagent;
washing means and discharge means, able respectively to wash the inside of the cell and to discharge the content of the cell, so as to prepare the cell for a subsequent reaction;
electronic processing means able to command and control, in the desired times and modes, at least the functioning of both the first dispenser element which dispenses the sample, of the microvalve dispensers which dispense the reagents, and of the mixer element, and also of the second dispenser element which sends the reacted sample for measurement, and of the washing means and discharge means, as a function of the type of reaction to be performed and the type of biological sample to be analyzed, so that their functioning is synchronized and automated according to pre-determined memorized reaction programs, selectable by an operator.

2. The reaction device as in claim 1, comprising heating means able to heat and thermostat the inside of the cell to a desired temperature value, correlated to the type of reaction to be carried out and according to a control signal received from the electronic processing means.

3. Device The reaction device as in claim 1, comprising blower means able to blow a selected fluid inside the cell, as a function of the reaction to be carried out and according to a control signal of the electronic processing means.

4. The reaction device as in claim 1, comprising a plurality of tanks to contain a determinate reagent, each of which is connected to a corresponding microvalve dispenser.

5. The reaction device as in claim 1, comprising pressure control means able to control and selectively vary the pressure inside the cell, as a function of the reaction to be carried out and according to a control signal of the electronic processing means.

6. The reaction device as in claim 1, wherein the electronic processing means comprises an electronic memory in which the desired reaction programs have been memorized and a microprocessor able to execute the programs.

7. The reaction device as in claim 6, wherein, according to a selected work program, the microprocessor is able to transmit command signals at least to activate/de-activate the functioning and/or regulate the quantities dispensed by the first and the second dispenser element and by the microvalve dispensers.

8. The reaction device as in claim 6, wherein, according to a selected work program, the microprocessor is able to transmit command signals at least to activate/de-activate the functioning and/or regulate the mixing power of the mixer element.

9. The reaction device as in claim 6, wherein the electronic processing means also comprises a timer by means of which the microprocessor is able to control and synchronize the functioning times both of the first dispenser element which dispenses the sample, the microvalve dispensers which dispense the reagents and the mixer element, and also the second dispenser element which sends the reacted sample for measurement, and also of the washing means and the discharge means.

10. The reaction device as in claim 1, comprising an electronic card, which is associated with a specific production batch of reagents, having memorizing means with at least a memorizing capacity, in which data for use of the reaction device are memorized, which data are read by reading means and transmitted to the processing means.

11. The reaction device as in claim 10, wherein said data comprise at least a univocal identification associated with the determinate and specific production batch of reagents, the reading by means of the reading means and the recognition of the univocal identification by the processing means providing consent to an operator to use the reaction device.

12. The reaction device as in claim 10, wherein, in said memorizing means, data are also memorized relating to the correct and necessary microvolumes to be dispensed for the reagent of the specific production batch of reagents, said data being acquired by reading the reading means by the processing means, which according to the data acquired is thus able to command at least the correct dispensing of the microvolumes of the reagents by the microvalve dispensers.

13. A method to subject, in sequence, a plurality of biological samples, liquid, semi-liquid or mixed, of analogous or different type, to one or more reactions for one or more determinate analyses, the method comprising the following steps:
a first step in which a predetermined quantity of the biological sample is introduced inside a reaction cell, by means of a first dispenser element;
a second step in which a determinate microvolume of a desired reagent is nebulized inside the cell by means of a plurality of microvalve dispensers, wherein the type of reagent and the amount of the microvolume of reagent are chosen according to the specific reaction to be carried out, selected from a group comprising: reactions for analyses of a chemical-biological type, such as immunological or coagulative reactions, or of a chemical-physical type;
a third step in which the biological sample is mixed with the selected reagent by means of a mixer element;
a fourth step in which a determinate quantity of the biological sample is sent to analytical measurement after reaction with the selected reagent by means of a second dispenser element;
a fifth step in which the inside of the cell is washed and the content of the cell is discharged, so as to prepare the cell for a subsequent reaction, respectively by means of washing means and discharge means;
wherein the functioning of both the first dispenser element which dispenses the sample, of the microvalve dispensers which dispense the reagents, and of the mixer element, and also of the second dispenser element which sends the reacted sample for measurement, and of the washing means and discharge means, is commanded and controlled, in the desired times and modes, by means of electronic processing means, as a function of the type of reaction to be performed and the type of biological sample to be analyzed, so that said functioning is synchronized and automated according to pre-determined memorized reaction programs, selectable by an operator.

14. The method as in claim 13, comprising a preliminary step prior to use in which a reading is made, by means of reading means, of data for the use of the reaction device, memorized in memorizing means with at least a memorizing capacity, of an electronic card, which is associated with a specific production batch of reagents, and said data are transmitted to the processing means.

15. The method as in claim 14, wherein said data comprise at least a univocal identification associated with the determinate and specific production batch of reagents, the reading of which, by means of the reading means, determines in the preliminary step the recognition of the univocal identification by the processing means which provides consent to an operator to use the reaction device.

16. The method as in claim 14, wherein, in said memorizing means, data are also memorized relating to the correct and necessary microvolumes to be dispensed for the reagent of the specific production batch of reagents, said data being acquired by reading the reading means in the preliminary step, by the processing means, which according to the data acquired commands at least the correct dispensing of the microvolumes of the reagents by the microvalve dispensers which is carried out in said second step.
